(19) United States
(12) Patent Application Publication
(10) Pub. No.: US 2011/0086060 A1
(43) Pub. Date: Apr. 14, 2011

(54) NOVEL COMPOSITIONS AND THEIR USE

(76) Inventors: Florence Bidamant, Mulhouse (FR); Dominik Imfeld, Munchenstein (CH); Peter Joller, Zurich (CH); Heidi Moser, Bubendorf (CH)

(21) Appl. No.: 12/933,961

(22) PCT Filed: Apr. 8, 2009
(86) PCT No.: PCT/EP09/54235

§ 371 Date: Dec. 20, 2010

Related U.S. Application Data
(60) Provisional application No. 61/044,105, filed on Apr. 11, 2008.

(51) Int. Cl. A61K 8/97; A61K 36/00; A61K 36/28; A61K 36/53; A61K 36/23; A61K 8/99; A61Q 19/00; A61P 29/00; A61P 17/02

(52) U.S. Cl. 424/195.17; 424/725; 424/764; 424/737; 424/745; 424/769; 424/744

(57) ABSTRACT

The present invention relates to a composition comprising panthenol, a collagen synthesis stimulating peptide and an anti-inflammatory extract. The compositions are particular useful for preventing and treating stretch marks.

### FIELD OF THE INVENTION

The present invention relates to a composition comprising panthenol, a collagen synthesis stimulating peptide and an anti-inflammatory extract. The compositions are particular useful for preventing and treating stretch marks.

### BACKGROUND OF THE INVENTION

Stretch marks commonly appear on the thighs, abdomen and buttocks and are marked by purplish-blue lines that fade into pale silvery-white lines. They are virtually impossible to completely and permanently remove. This is because they are the marks of damaged fibers under the skin. The whole process is triggered by ongoing inflammation processes in the dermis. The dermal collagen and elastin fiber-matrix are damaged and dehydrated leaving visible marks or scars. A stretch mark is actually scar tissue that is visible on the outer layer of skin. Fibers are damaged in various ways, but the two most common examples are when rapid weight gain stretches the skin (i.e.: growth spurts or pregnancy), or through the muscle fibers thickening (i.e.: bodybuilding). The actual stretch mark is the consequence of the dermis stretching to the point of breaking cell-to-cell contact and the fibers of the basal membrane. The initial damage appears in the form of red lines, fading into a pale-white-colored scar once it's healed over. Stretch marks can become more evident over time, as they can change color and also become more prominent when a significant amount of weight is lost. Though stretch marks can rarely be removed completely, there are many treatments available for the purpose of improving the appearance of existing stretch marks including cosmetic surgery, laser treatments and dermabrasion. However, these treatments are rather costly and downright painful for some, with no assurance that it will indeed work. Topical preparations containing retinoids, alpha hydroxy acids or salicylic acid are also known to reduce stretch marks, however these ingredients may induce skin irritation, redness and flaking and even an increased photosensitivity to the sun. Furthermore, their effectiveness is still not sufficient.

Thus, there is a growing demand for topical preparations which can be effectively used for minimizing or even eliminating stretch marks. At the same time such topical preparations should preferably exhibit no skin irritation. Thus, identifying a composition which overcomes the drawbacks of the prior art having at the same time a low irritation or even an anti-inflammatory potential would be of significant commercial interest.

### SUMMARY OF THE INVENTION

The invention relates to a composition comprising panthenol, an anti-inflammatory plant extract and a collagen synthesis stimulating peptide.

In another embodiment, the invention relates to a topical preparation comprising an effective amount of a composition comprising panthenol, an anti-inflammatory plant extract and a collagen synthesis stimulating peptide in combination with a cosmetically acceptable carrier. Furthermore, the invention relates to a method of decreasing the actual stretch marks, protecting stretched skin fibers, decreasing the depth of indented surfaces, increasing smooth surfaces, increasing skin thickness and firmness as well as density, increases stimulation of collagen synthesis, moisturizing the skin, reducing inflammation and accelerating skin renewing and wound healing said method comprising the step of applying an effective amount of a topical preparation comprising panthenol, an anti-inflammatory plant extract and a collagen synthesis stimulating peptide in combination with a cosmetically acceptable carrier to the skin of a subject in need of such a treatment.

In another aspect, the invention relates to a method of treatment or co-treatment of stretch marks, said method comprising the step of applying an effective amount of a topical preparation comprising panthenol, an anti-inflammatory plant extract and a collagen synthesis stimulating peptide in combination with a cosmetically acceptable carrier to the skin of a subject in need of such a treatment.

### DETAILED DESCRIPTION OF THE INVENTION

Collagen synthesis stimulating peptides are mikropeptides which are able to stimulate the development of new collagen, elastin and/or glucosaminoglycans. For example, the tripeptide N2-(1-oxohexadecyl)-L-lysyl-L-valyl-L-lysine or a salt thereof, in particular the bistrifluoroacetate salt is capable of stimulating Collagen type I synthesis by optimally mimicking the thrombospondin-1 sequence Arg-Phe-Lys which converts latent TGFbeta1 into its active form. TGFbeta1 induces the collagen and elastin synthesis in the skin.

Anti-inflammatory plant extracts play an important role in the inflammation process by down-regulating the important pro-inflammatory cytokines like IL-1α, IL-1β, IL-8 and TNF-α. Such extracts are well known for their excellent healing properties on skin.

For cosmetic applications Panthenol (also known as Dexpanthenol or Pantothenol) is known as a humectant, emollient and moisturizer. Furthermore it reduces itchiness of the skin as well as has an anti-inflammatory effect.

Surprisingly it has now been found that a composition comprising a collagen synthesis stimulating peptide, an anti-inflammatory plant extract and panthenol effectively and even synergistically protects stretched fibroblasts. Furthermore, it has been found that topical preparations comprising an effective amount of a composition comprising a collagen synthesis stimulating peptide, an anti-inflammatory extract and panthenol are suitable for the treatment and the prevention of stretch mark related skin damage and stretch marks as such.

Thus, the invention relates to a composition comprising panthenol, an anti-inflammatory plant extract and a collagen synthesis stimulating peptide.

Preferred collagen synthesis stimulating peptides refers to synthetic peptides consisting of 3 to 10, in particular 3 to 5 amino acids linked together which are attached to a fatty acid to enhance the oil solubility. Preferably, the fatty acid is Palmitic acid. In all embodiments of the invention, preferred collagen synthesis stimulating peptides include the tripeptide N2-(1-oxohexadecyl)-L-lysyl-L-valyl-L-lysine or a salt thereof, in particular the bistrifluoroacetate salt as well as the pentapeptide Palmitoyl-Lys-Thr-Thr-Lys-Ser or a salt thereof. The most preferred collagen synthesis stimulating peptide according to the invention is the tripeptide N2-(1-oxohexadecyl)-L-lysyl-L-valyl-L-lysine or a salt thereof, in particular the bistrifluoroacetate salt of N2-(1-oxohexadecyl)-L-lysyl-L-valyl-L-lysine.

In particular, the invention relates to a composition comprising panthenol, an anti-inflammatory plant extract and a collagen synthesis stimulating peptide selected from the tripeptide N2-(1-oxohexadecyl)-L-lysyl-L-valyl-L-lysine or a salt thereof and/or the pentapeptide Palmitoyl-Lys-Thr-Thr-Lys-Ser or a salt thereof.

Preferred anti-inflammatory plant extracts in all embodiments of the invention include extracts obtainable from Calendula officinalis, Leontopodium alpinum, Echinacea purpurea, Malva sylvestris, Thymus vulgaris, Peucedanum ostruthium, and Marrubium vulgare L.

In another aspect the present invention relates to a composition comprising
(a) at least 10 wt.-% of panthenol, preferably 10-50 wt.-%,
(b) at least 0.005 wt.-% of a collagen synthesis stimulating peptide or a salt thereof, preferably 0.005-10 wt.-% and
(c) at least 0.01 wt.-% of an anti-inflammatory plant extract, preferably 0.01-40 wt.-%.

In a preferred embodiment the composition comprises
(a) at least 20 wt.-% of panthenol, preferably 25-40 wt.-% and
(b) at least 0.01 wt.-% of a collagen synthesis stimulating peptide or a salt thereof, preferably 0.01-1 wt.-% and
(c) at least 0.1 wt.-% of an anti-inflammatory plant extract, preferably 0.1-5 wt.-%.

In a particular embodiment, the invention relates to a composition comprising panthenol, a Marrubium vulgare extract and N2-(1-oxohexadecyl)-L-lysyl-L-valyl-L-lysine or a salt thereof.

In another particular aspect the present invention relates to a composition comprising
(a) at least 10 wt.-% of panthenol, preferably 10-50 wt.-%,
(b) at least 0.005 wt.-% of N2-(1-oxohexadecyl)-L-lysyl-L-valyl-L-lysine or a salt thereof preferably 0.005-10 wt.-% and
(c) at least 0.01 wt.-% of a Marrubium vulgare extract, preferably 0.01-40 wt.-%.

In a preferred embodiment the composition comprises
(a) at least 20 wt.-% of panthenol, preferably 25-40 wt.-% and
(b) at least 0.01 wt.-% of N2-(1-oxohexadecyl)-L-lysyl-L-valyl-L-lysine or a salt thereof preferably 0.01-1 wt.-% and
(c) at least 0.1 wt.-% of a Marrubium vulgare extract, preferably 0.1-5 wt.-%.

Most preferred according to the invention is a composition comprising

30 wt.-% of panthenol,
0.02 wt.-% of N2-(1-oxohexadecyl)-L-lysyl-L-valyl-L-lysine or a salt thereof and 0.4 wt.-% of a Marrubium Vulgare extract.

In a particular preferred embodiment the compositions according to the present invention further comprises other usual cosmetic additives whereas the total amount adds up to 100 wt.-%. Preferably, the further cosmetic additives are selected from
alcohols, especially lower alcohols, preferably ethanol and/or isopropanol, low diols or polyols and their ethers, preferably propyleneglycol, glycerin, ethyleneglycol, ethyleneglycol monoethyl- or monobutylether, propyleneglycol monomethyl- or -monoethyl- or -monobutylether, diethyleneglycol monomethyl- or monoethylether and analogue products in particular glycerin and/or
water and/or
preservatives such as Potassium Sorbate, Sodium benzoate, Methyl-, Ethyl-, Propyl-, Butylparabens preferably Potassium Sorbate and/or Sodium Benzoate.

In a specific embodiment the invention relates to a composition consisting of

### About 30 wt.-% of Panthenol,

About 0.40 wt.-% of a Marrubium vulgare Extract,
About 0.02 wt.-% of N2-(1-oxohexadecyl)-L-lysyl-L-valyl-L-lysine or a salt thereof,

### About 28 wt.-% of Water,

### About 41 wt.-% of Glycerine,

### About 0.20 wt.-% of Sodium Benzoate and

### About 0.20 wt.-% of Potassium Sorbate.

So that all ingredients sum up to 100 wt.-%.

The compositions according to the invention can be used as such in the desired application form such as e.g. in topical preparations. However, the compositions according to the invention are also suitable to be encapsulated in nanoparticles such as liposomes, nanosomes, cyclodextrins, which subsequently may be incorporated into the desired application form.

In all embodiments of the invention the N2-(1-oxohexadecyl)-L-lysyl-L-valyl-L-lysine or a salt thereof is the bistrifluoroacetate salt of N2-(1-oxohexadecyl)-L-lysyl-L-valyl-L-lysine (listed in the CTFA Dictionary as Palmitoyl Tripeptide-5) and which is commercially available under the trade name SYN®-COLL at DSM Nutritional Products Ltd. Branch Pentapharm.

The pentapeptide Palmitoyl-Lys-Thr-Thr-Lys-Ser (listed in the CTFA Dictionary as Palmitoyl pentapeptide-3) is commercially available as Matrixyl® at Thalgo cosmetic GMBH.

The plant extracts obtainable from Calendula officinalis, Leontopodium alpinum, Echinacea purpurea, Melva sylvestris, Thymus vulgaris, Peucedanum ostruthium and Marrubium vulgare L. are well known to a person skilled in the art and commercially available e.g. at Mountain Rose Herbs or at DSM Nutritional Products Ltd., Branch Pentapharm (inter alia under the trade names MALVA®AO, CALENDULA® AO, LINUM®AO, THYMUS®AO, SCUTELLARIA®AO, IMPERATORIA®AO).

The term Marrubium vulgare extract as used herein refers to an extract of the aerial parts, in particular the stems and leaves of Marrubium vulgare L. also known as Horehound. Such extracts are obtainable by extraction of the aerial parts such as the stems and leaves with a suitable solvent such as e.g. selected from the group consisting of water, alcohols preferably containing from 1 to 4 carbon atoms, such as methanol, ethanol or propanol, an aqueous-alcoholic mixture of these alcohols, chlorinated solvents containing 1 or 2 carbon atoms, such as chloroform or dichloromethane, and organic esters preferably containing 3 to 6 carbon atoms, such as ethyl acetate in particular a mixture from water/ethanol is used. The extraction is carried out at temperatures between room temperature and the boiling point of the solvent used for the extraction. A valuable extraction technique is the so-called Soxhlet extraction technique. However, it is also possible simply to carry out the extraction at normal atmospheric pressure for 2 to 24 h, if appropriate after the plant material has been left to macerate for 2 to 4 h in the cold extraction solvent. When the extraction is complete, the phase containing the extract is filtered and optionally concentrated to the final concentration needed and/or evaporated to complete dryness under reduced pressure or by lyophilization. The concentrate may be further formulated using appropriate solvents and/or cosmetically usual additives such as glycerin or any type of glycols such as e.g. propyleneglycol or 1,3-butanediol and/or preservatives.

The ratio of the plant material to the extraction agent is not critical but is generally between 1:5 and 1:20 parts per weight, preferably 1:10.

In all embodiments of the invention, the Marrubium vulgare extract is preferably an aqueous/ethanolic extract of the aerial parts of Marrubium vulgare L.

In all embodiments of the invention, a particular preferred Marrubium vulgare extract is ALPAFLOR® MARRUBIUM AO from the ALP®-ORGANIC line available at DSM Nutritional Products Ltd., Branch Pentapharm.

The term Panthenol as used in this context refers to D-Panthenol, DL-Panthenol, Panthenyltriacetat as well as to Ethyl panthenol. In all embodiments of the invention, preferably D-Panthenol is used which is e.g. commercially available as D-Panthenol 75 L from DSM Nutritional Products Ltd.

The present invention also pertains to topical preparations such as cosmetic, pharmaceutical and veterinary medical preparations comprising a composition according to the invention.

Thus, the present invention also relates to topical preparations comprising an effective amount of a composition according to the invention and a cosmetically acceptable carrier.

The term effective amount refers to an amount of at least 0.01 wt.-%. More preferably an amount of 0.1 to 10 wt.-%, in particular 1 to 3 wt.-% based on the total weight of the preparation is used.

The term “topical preparation” as used herein refers in particular to a cosmetic preparation that can be topically applied to mammalian keratinous tissue such as e.g. human skin or hair, particularly human skin.

The term “cosmetic preparation” as used in the present application refers to cosmetic compositions as defined under the heading “Kosmetika” in Römpp Lexikon Chemie, 10th edition 1997, Georg Thieme Verlag Stuttgart, New York as well as to cosmetic preparations as disclosed in A. Domsch, “Cosmetic Preparations”, Verlag für chemische Industrie (ed. H. Ziolkowsky), 4^{th} edition, 1992.

The term cosmetically acceptable carrier refers to all carriers and/or excipients and/or diluents conventionally used in topical preparations

Preferably, the topical preparations according to the present invention are in the form of a suspension or dispersion in solvents or fatty substances, or alternatively in the form of an emulsion or micro emulsion (in particular of O/W- or W/O-type), PIT-emulsion, multiple emulsion (e.g. O/W/O- or W/O/W-type), pickering emulsion, hydrogel, alcoholic gel, lipogel, one- or multiphase solution or vesicular dispersion or other usual forms, which can also be applied by pens, as masks or as sprays. If the topical preparation is or comprises an emulsion it can also contain one or more anionic, nonionic, cationic or amphoteric surfactant(s).

Preferred topical preparations according to the invention are skin care preparations, and functional preparations.

Examples of skin care preparations are, in particular, body oils, body lotions, body gels, treatment creams, skin protection ointments, shaving preparations, such as shaving foams or gels, skin powders such as baby powder, moisturizing gels, moisturizing sprays, revitalizing body sprays, cellulite gels, face and/or body moisturizers, facial and/or body cleansers, face masks, anti acne preparations and/or peeling preparations.

Examples of functional preparations are cosmetic or pharmaceutical preparations containing active ingredients such as hormone preparations, vitamin preparations, vegetable extract preparations, anti-ageing preparations, and/or antimicrobial (antibacterial or antifungal) preparations without being limited thereto.

Topical preparations in accordance with the invention can be in the form of a liquid, lotion, a thickened lotion, a gel, a cream, a milk, an ointment, a paste, a powder, a make-up, or a solid tube stick and can be optionally be packaged as an aerosol and can be provided in the form of a mousse such as a aerosol mousse, a foam or a spray foam, a spray, a stick, a plaster, a cleanser, a soap, a wipe or a lyophilizate (such as the Pentapharm Dual Vial system).

In accordance with the present invention, the topical preparation contains a composition according to the invention, optionally in combination with further ingredients such as ingredients for skin lightening; tanning prevention; treatment of hyperpigmentation; preventing or reducing acne, wrinkles, lines, atrophy and/or inflammation; as well as topical anesthetics; antimicrobial and/or antifungal agents; chelators and/or sequestrants; anti-cellulites and slimming (e.g. phytanic acid), firming, moisturizing and energizing, self tanning, soothing, as well as agents to improve elasticity and skin barrier and/or further UV-filter substances and carriers and/or excipients or diluents conventionally used in topical preparations. If nothing else is stated, the excipients, additives, diluents, etc. mentioned in the following are suitable for topical preparations according to the present invention. The necessary amounts of the cosmetic and dermatological adjuvants and additives can, based on the desired product, easily be determined by the skilled person.

The cosmetically active ingredients useful herein can in some instances provide more than one benefit or operate via more than one mode of action.

The topical preparations according to the present invention may contain further cosmetically active ingredients. Examples of cosmetically active ingredients comprise peptides (e.g., Matrixyl™ [pentapeptide derivative], one or both of the peptides contained in SYN®-TACKS from DSM Nutritional Products Ltd., Branch Pentapharm), oligopeptides, wax-based synthetic peptides and palmitoyl-oligopeptide), iodopropyl butylcarbamate, glycerol, urea, guanidine (e.g. amino guanidine); vitamins and derivatives thereof such as vitamin C (ascorbic acid), vitamin A (e.g., retinoid derivatives such as retinyl palmitate or retinyl propionate), vitamin E (e.g., tocopherol acetate), vitamin B₃ (e.g. niacinamide) and vitamin B₅ (e.g. panthenol), vitamin B₆ and vitamin B₁₂, biotin, folic acid; anti-acne actives or medicaments (e.g. resorcinol, salicylic acid, and the like); antioxidants (e.g. phytosterols, lipoic acid); flavonoids (e.g. isoflavones, phytoestrogens); skin soothing and healing agents such as Aloe vera extract, allantoin and the like; agents suitable for aesthetic purposes such as essential oils, fragrances, skin sensates, opacifiers, aromatic compounds (e.g., clove oil, menthol, camphor, eucalyptus oil, and eugenol and their derivatives), desquamatory actives, hydroxy acids such as AHA acids, BHA acids, poly unsaturated fatty acids, radical scavengers, farnesol, antifungal actives in particular bisabolol, alkyldiols such as 1,2-pentanediol, hexanediol or 1,2-octanediol, phytol, polyols such as phytanetriol, ceramides and pseudoceramides, amino acids, protein hydrolysates, polyunsaturated fatty acids, plant extracts like kinetin, DNA or RNA and their fragmentation products, carbohydrates, conjugated fatty acids, carnitin, carnosine, biochinones, phytofluen, phytoen, and their corresponding derivatives, co-enzyme Q10/ubiquinone), anti-oxidants, preferably (−)-epigallocatechin gallate (EGCG), hydroxytyrosol, and/or olive extract, shea butter, algae extract, cocoa butter, aloe extract, elastin and GAG booster without being limited thereto.

Preferred examples of cosmetically active ingredients are vitamin C (ascorbic acid) and/or its derivatives (e.g. ascorbyl phosphate such as Stay C (sodium ascorbyl monophosphate) from DSM Nutritional Products Ltd.), vitamin A and/or its derivatives (e.g., retinoid derivatives such as retinyl palmitate or retinyl propionate), vitamin E and/or its derivatives (e.g., tocopherol acetate), vitamin B₆, vitamin B₁₂, biotin, co-enzyme Q10, EGCG, hydroxytyrosol and/or olive extract, shea butter, algae extract, cocoa butter, aloe extract, jojoba oil, echinacea extract, elastin and GAG booster in particular vitamin E and/or its derivatives, shea butter, algae extract, cocoa butter, aloe extract, elastin and GAG booster, vitamin C (ascorbic acid) and/or its derivatives and/or vitamin A and/or its derivatives. The additional cosmetically active ingredient is typically included in an amount of at least 0.001 wt. % based on the total weight of the topical preparation. Generally, an amount of about 0.001 wt. % to about 30 wt. %, preferably from about 0.001 wt. % to about 10 wt. % of an additional cosmetically active agent is used.

Vitamin C (ascorbic acid) and/or its derivatives in particular ascorbyl phosphate such as Stay C (sodium ascorbyl monophosphate) is preferably used in the topical preparations according to the invention in an amount of 0.1-5 wt.-% in particular 0.1-2 wt.-%.

Shea butter is preferably used in the topical preparations according to the invention in an amount of 0.5-10 wt.-%, in particular 0.5-5 wt.-%.

Algae extract is preferably used in the topical preparations according to the invention in an amount of 0.1-10 wt.-%, in particular 0.5-1 wt.-%.

Aloe extract is preferably used in the topical preparations according to the invention in an amount of 0.1-10 wt.-%, in particular 0.5-1 wt.-%.

Elastin is preferably used in the topical preparations according to the invention in an amount of 0.01-10 wt.-%, preferably 0.01-1 wt.-%

GAG booster is preferably used in the topical preparations according to the invention in an amount of 0.001-10 wt.-%.

A vitamin E derivative for use in the present invention is tocopheryl acetate. Tocopheryl acetate may be present in the topical preparations in an amount from about 0.05 wt.-% to about 25 wt.-%, in particular 0.05 wt.-% to 5 wt.-%. Another vitamin E derivative of interest is tocopheryl linoleate. Tocopheryl linoleate may be present in the skin care composition in an amount from about 0.05 wt.-% to about 25 wt.-% in particular 0.05 wt.-% to 5 wt.-%. Please verify

Vitamin A and/or its derivatives in particular retinoid derivatives such as retinyl palmitate or retinyl propionate is preferably used in the topical preparations according to the invention in an amount of 0.01-5 wt.-%, in particular 0.01-0.3 wt.-%

Cocoa butter is preferably used in the topical preparations according to the invention in an amount of 0.5-5 wt.-%.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention are e.g. described in the CTFA Cosmetic Ingredient Handbook, Second Edition (1992) without being limited thereto.

The necessary amounts of the cosmetic and dermatological adjuvants and additives can—based on the desired product—easily be chosen by a skilled person in this field and will be illustrated in the examples, without being limited hereto.

The usual cosmetic adjuvants and additives such as e.g. emulsifiers, thickeners, surface active ingredients and film formers can show synergistic effects which can be determined by the expert in the field with normal trials, or with the usual considerations regarding the formulation of cosmetic composition.

Which amount of the topical preparation has to be applied, depends on the concentration of the active ingredient(s) in the product and the desired cosmetic effect(s). A typical “leave-on” composition like a skin care emulsion or a functional preparation, for example, is usually applied in an amount of about 0.5 to about 2 mg per cm² skin. The applied amount is normally not critical, and the desired effect(s) may be achieved by using more of the composition, repeating the application of the composition and/or applying a composition which contains more of the active ingredient(s).

By “‘leave-on’ composition” as used herein a topical preparation is meant which after having applied to the skin, is not removed intentionally. It is preferably left on the skin for a period of at least about 15 minutes, more preferably at least about 30 minutes, even more preferably at least about 1 hour, most preferably for at least several hours, e.g. up to about 12 hours.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The compositions according to the invention are preferably formulated an oil-in-water or water-in-oil emulsion, water-in-silicone or silicone-in-water emulsion or as an aqueous serum or aqueous gel.

The cosmetic and/or dermatological compositions according to the invention have a pH in the range of 3-10, preferably in the range of pH of 4-8, most preferred in the range of pH 4-6.

The topical preparations according to the invention or in particular useful for decreasing the actual stretch marks, protecting stretched skin fibers, decreasing the depth of indented surfaces, increasing smooth surfaces, increasing skin thickness and firmness as well as density, increases stimulation of collagen synthesis, moisturizing the skin, reducing inflammation and accelerating skin renewing and wound healing.

Thus, in another embodiment, the invention also relates to a method of decreasing the actual stretch marks, protecting stretched skin fibers, decreasing the depth of indented surfaces, increasing smooth surfaces, increasing skin thickness and firmness as well as density, increases stimulation of collagen synthesis, moisturizing the skin, reducing inflammation and accelerating skin renewing and wound healing said method comprising the step of applying an effective amount of a topical preparation with all the definition and preferences as given above to the skin of a subject in need of such a treatment. In particular, the invention relates to a method of treatment or co-treatment of stretch marks, said method comprising the step of applying an effective amount of a topical preparation with all the definition and preferences as given above to the skin of a subject in need of such a treatment. The term treatment or co-treatment as used in the present invention includes also a proactive use of the topical preparations in order to prevent the formation of stretch marks.

An effective amount of a topical preparation with the definitions and preferences as given above in these methods refers to an amount necessary to obtain a physiological effect. The physiological effect may be achieved by one single dose or by repeated doses. The dosage administered may, of course, vary depending on known factors, such as the physiological characteristics of the particular composition and its mode and route of administration; the age, health and weight of the recipient; the nature and extent of the symptoms; the kind of concurrent treatment; the frequency of treatment; and the effect desired and can be adjusted by a person skilled in the art.

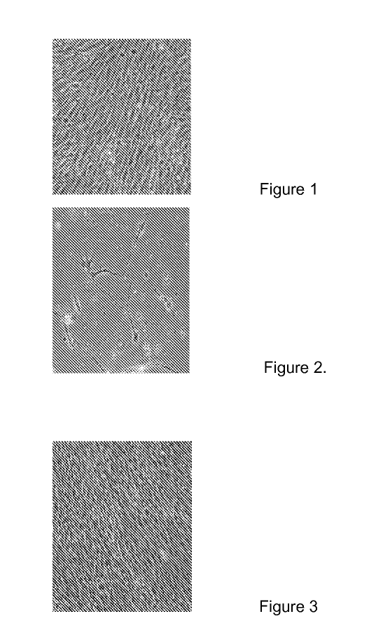

FIG. 1 shows the control (culture of human fibroblasts in medium only), not stretched and not protected.

FIG. 2 shows the untreated stretched fibroblasts.

FIG. 3 shows the stretched fibroblasts, treated with 0.05 vol.-% of the preparation of table 1.

The following examples are provided to further illustrate the compositions and effects of the present invention. These examples are illustrative only and are not intended to limit the scope of the invention in any way.

### Example 1

### In Vitro Efficacy

The composition disclosed in table 1 has been in tested in cell culture. Skin stretching process has been simulated in a glass/acryl box by stretching a monolayer of fibroblasts cultures. Monolayer fibroblast cultures were placed on cell culture plates with a flexible silicone bottom and stretched over glass hemispheres attached to an acryl support. With this test equipment equibiaxial stretching of the cell monolayer's up to +41% is possible. The fibroblast cultures were treated with 0.05 vol.-% of a composition according to the invention as outlined in table 1. The cultures were morphologically examined at the different stages of stretching. Cell number and morphology under the microscope were the end-points to analyze the effects of our active ingredients. Simultaneously cell culture medium was collected at different time points and quantitatively analyzed for 7 cytokines and 4 Matrix Metallo Proteinases (MMPs) in a multiplex bead array system (Luminex¹⁰⁰™)

As can be seen from FIGS. 1 to 3, the composition according to the invention has an excellent protective effect on fibroblasts.

This effect has been shown to be synergistic by comparison with fibroblast cultures which have been treated with 0.05 vol.-% of the single active ingredients, i.e. Panthenol, a Marrubium vulgare extract or Palmitoyl Tripeptide-5 as described above.

The results of the cytokines measurements (protein quantity) is shown below:

| | IL-1α | IL-1β | IL-8 | TNF-α | MMP-2 | MMP-3 | MMP-9 | MMP-13 |
|---|---|---|---|---|---|---|---|---|
| Just after stretching | −50% | −41% | −18% | −7% | −5% | +10% | −8% | −19% |
| (10 minutes) | | | | | | | | |
| 2 hours after | −50% | −44% | −40% | −20% | −14% | −13% | −14% | −28% |
| stretching | | | | | | | | |
| 6 hours after | −76% | −74% | −87% | −49% | −66% | −72% | −70% | −77% |
| stretching | | | | | | | | |

TABLE 1
composition for in vitro-test

| INCI Name | Wt.-%-Anteile |
|---|---|
| Panthenol¹ | 30.00 |
| Marrubium vulgare extract² | 0.40 |
| Palmitoyl Tripeptide-5³ | 0.02 |
| Water | 28.00 |
| Glycerin | Ad 100.00 |
| Sodium Benzoate | 0.20 |
| Potassium Sorbate | 0.20 |

¹D-Panthenol 75-L from DSM Nutritional Products Ltd
²ALPAFLOR ® Marrubium AO from DSM Nutritional Products Ltd., Branch Pentapharm
³SYN ®-COLL from DSM Nutritional Products Ltd., Branch Pentapharm

### Example 2

### In Vivo Study

The anti stretch mark effect of the topical preparation disclosed in table 2 has been measured in a 3 month clinical study. 30 female caucasian volunteers aged 18 or more with recent/red stretch marks have been treated for 84 days. The preparation given in table 2 was applied twice daily by the volunteers themselves in their own manner. The stretch mark length and width (centrimetric measurement) and the dermis density has been measured at day 0 and after 84 days.

TABLE 2
in vivo preparation

| Ingredients | INCI Name | wt.-% |
|---|---|---|
| Water | Aqua | Ad 100 |
| Glycerin | Glycerin | 3.00 |
| Phenonip | Phenoxyethanol, Methylparaben, | 0.80 |
| | Ethylparaben, Butylparaben, | |
| | Propylparaben, Isobutylparaben | |
| Berry Wax 6290 | Rhus Verniciflua Peel Wax | 0.40 |
| Rice Bran Wachs | Oryza sativa (Rice) Bran Wax | 0.10 |
| Jojoba Oil, sweet | Simmondsia chinensis (Jojoba) | 5.00 |
| | Seed Oil | |
| Lexfeel 7 | Neopentyl Glycol Diheptanoate | 7.00 |
| Novemer EC-1 | Acrylates/Acrylamide Copolymer, | 2.00 |
| | Mineral Oil, Polysorbate 85 | |
| Citric Acid 50% | Citric Acid | 4 Tr. |
| Composition | Water, Glycerin, Palmitoyl | 3.00 |
| according to | tripeptide-5, Panthenol, | |
| table 1 | Marrubium vulgare extract | |

As can be seen in table 3, the stretch marks length and width was significantly reduced. Furthermore, the dermis density increased significantly.

TABLE 3
in vivo results

| | |
|---|---|
| Reduction of Stretch marks' length | Up to −13.50% |
| Reduction of Stretch marks' width | Up to −22.20% |
| Increase of dermis' density | Up to 29.00% |

Furthermore, the volunteers evaluated the effect of the treatment based on a questionnaire as outlined in table 4.

TABLE 4
questionnaire

| | Percentage of volunteers |
|---|---|
| Parameter | with positive effect |
| Decrease in stretch marks' length | 92% |
| Decrease in stretch marks' width | 92% |
| Decrease in stretch marks' color | 92% |
| Skin on stretch marks suppler | 69% |
| Disappearance of stretch marks | 31% |
| on the treated zone | |

As can be retrieved from table 4, all volunteers attributed a significant improvement to the treated area.

### Example 3

### Light W/O Emulsion

| Phase | Ingredients | INCI Name | wt.-% |
|---|---|---|---|
| A | Isolan | Polyglyceryl-4 | 3.00 |
| | | Diisostearate/Polyhydroxystearate/ | |
| | | Sebacate | |
| | Tegosoft | Diethylhexyl Carbonate | 7.00 |
| | DEC | | |
| | DC 345 | Cyclopentasiloxane | 8.80 |
| | Shea Butter | Butyrospermum parkii | 3.00 |
| | | Hydrogenated Castor Oil | 0.10 |
| | Retinol | Retinol Palmitate | 0.50 |
| B | Glycerin | Glycerin | 2.00 |
| | Vitamin C | Aqua, Ascorbic Acid | 0.30 |
| | | Magnesium Sulfate Heptahydrate | 1.50 |
| | Wasser | Aqua | Ad 100 |
| C | Composition | Water, Glycerin, Palmitoyl | 3.00 |
| | according to | tripeptide-5, Panthenol, | |
| | table 1 | Marrubium vulgare extract | |
| | Parfum | Fragrance/Perfum | q.s. |
| | Blue | FD & C Blue 1 | q.s. |
| | Red | D&C Red 40 | q.s. |
| | NaOH | Sodium hydroxide | q.s. |

### Example 4

### Self Tanning Lotion

| Phase | Ingredients | INCI Name | wt.-% |
|---|---|---|---|
| A | Abil EM 90 | Cetyl PEG/PPG-10/1 | 2.00 |
| | | Dimethicone | |
| | Isopropylstearate | Isopropyl Stearate | 10.00 |
| | Mineral Oil | Mineral Oil | 10.00 |
| | Shea Butter | Butyrospermum parkii | 4.50 |
| | Microcristalline | Microcristalline Wax | 0.40 |
| | Wax | | |
| | Kakao Butter | Theobroma cacao (Cocoa) | 2.00 |
| | | Seed Butter | |
| | DL Tocopherol- | Tocopherol Acetate | 0.50 |
| | acetat | | |
| | Retinol | Retinol Palmitate | 0.50 |
| B | Glycerin | Glycerin | 2.00 |
| | C*Pharmsorbidex | Sorbitol, Aqua | 3.00 |
| | NC 16205 | | |
| | Vitamin C | Aqua, Ascorbic Acid | 0.30 |
| | | Sodium Chloride | 0.50 |
| | Wasser | Aqua | Ad 100 |
| C | PEPHA ®- | Aqua, Pullulan, Algae Extract, | 2.00 |
| | TIGHT | Phenoxyethanol, Sodium | |
| | | Benzoate, Potassium Sorbate | |
| | DHA | Dihydroxyacetone | 5.00 |
| | Erythrulose | Erythrulose, Aqua | 2.00 |
| | Composition | Water, Glycerin, Palmitoyl | 3.00 |
| | according to | tripeptide-5, Panthenol, | |
| | table 1 | Marrubium vulgare extract | |
| | Euxyl PE 9010 | Phenoxyethanol, Ethylglycerin | 0.80 |
| D | Parfum | Fragrance/Perfum | q.s. |
| | FD & C Blue 1 | FD & C Blue 1 | q.s. |
| | NaOH | Sodium Hydroxide | q.s. |

### Example 5

### O/W Emulsion with Cooling Effect

| Phase | Ingredients | INCI Name | wt.-% |
|---|---|---|---|
| A | Deionised Water | Aqua | Ad 100 |
| | Glycerin | Glycerin | 2.00 |
| | Carbopol EDT | Acrylates/C10-30 Alkyl | 0.40 |
| | 2020 | Acrylate Crosspolymer | |
| B | Lexol EHS | Ethylhexyl Stearate | 3.00 |
| | Cetiol OE | Dicaprylyl Ether | 5.00 |
| | Macadamianussöl | Macadamia ternifolia Seed Oil | 5.00 |
| | Procol PSA-15 | PPG-15 Stearyl Ether | 3.00 |
| | Keltrol CG-F | Xanthan gum | 0.20 |
| | Euxyl PE 9010 | Phenoxyethanol, Ethylglycerin | 0.80 |
| C | Composition | Water, Glycerin, Palmitoyl | 3.00 |
| | according to | tripeptide-5, Panthenol, | |
| | table 1 | Marrubium vulgare extract | |
| | Frescolat ML | Menthyl Lactate | 1.00 |
| | Parfum | Fragrance, Perfum | 0.10 |
| | NaOH | Sodium Hydroxide | q.s. |

### Example 6

### O/W Emulsion for Pregnant Woman

| Phase | Ingredients | INCI Name | wt.-% |
|---|---|---|---|
| A | Olivem 1000 | Cetearyl Olivate (and) Sorbitan | 4.00 |
| | | Olivate | |
| | Kaffee Butter | Coffea arabica, Hydrogenated | 4.50 |
| | | Vegetable Oil | |
| | Kakao Butter | Theobroma cacao (Cocoa) | 4.00 |
| | | Seed Butter | |
| | Shea Butter | Butyrospermum parkii | 4.50 |
| | Jojobaöl | Simmondsia chinensis (Jojoba) | 5.00 |
| | | Seed Oil | |
| | Sweet almond Oil | Prunus amygdalus dulcis | 5.00 |
| | | (Sweet Almond) Oil | |
| | DL Tocopherol- | Tocopherol Acetate | 0.50 |
| | acetat | | |
| B | Glycerin | Glycerin | 3.00 |
| | Sisterna L70-C | Aqua, Sucrose Laurate, Alcohol | 3.00 |
| | Euxyl 9010 | Phenoxyethanol, Ethylglycerin | 0.80 |
| | Keltrol | Xanthan gum | 0.40 |
| | C*Pharmsorbidex | Sorbitol, Aqua | 1.00 |
| | NC 16205 | | |
| | Wasser ad 100 | Aqua | 60.20 |
| C | ALPAFLOR ® | Glycerin, Aqua, Echinacea | 1.00 |
| | Echinacea AO | purpurea (Echinacea) | |
| | | Extract, Potassium Sorbate, | |
| | | Sodium Benzoate | |
| | Composition | Water, Glycerin, Palmitoyl | 3.00 |
| | according to | tripeptide-5, Panthenol, | |
| | table 1 | Marrubium vulgare extract | |
| D | Parfum | Fragrance/Perfum | 0.10 |
| | NaOH | Sodium hydroxide | q.s. |

### Example 7

### Anti Stretchmarks Body Lotion for Teens

| Phase | Ingredients | INCI Name | % Wt |
|---|---|---|---|
| A | Pemulen TR-1 | Acrylates/C10-30 Alkyl | 0.60 |
| | | Acrylate Crosspolymer | |
| | Lexol EHS | Ethylhexyl Stearate | 8.00 |
| | Rose Hip Oil | Rosa Moschata | 10.00 |
| | DL Tocopherol- | Tocopheryl Acetate | 0.50 |
| | acetat | | |
| | Euxyl 9010 | Phenoxyethanol, Ethylglycerin | 0.80 |
| B | Glycerin 86% | Glycerin | 2.00 |
| | Nicotinsäureamid | Niacinamide | 0.20 |
| | Kaffeine | Coffein | 0.20 |
| | Wasser | Aqua | ad 100 |
| C | NaOH, 10% | Sodium Hydroxide | 0.35 |
| D | Composition | Water, Glycerin, Palmitoyl | 3.00 |
| | according to | tripeptide-5, Panthenol, | |
| | table 1 | Marrubium vulgare extract | |
| E | Parfum | Fragrance | 0.20 |
| | Rot 1% | D&C Red 40 | 0.50 |
| | Blau 0.1% | FD&C Blue 1 | 0.20 |
| | Gelb 1% | FD&C Yellow 6 | 0.30 |

### Example 8

### O/W Emulsion

| Phase | Ingredients | INCI Name | wt.-% |
|---|---|---|---|
| A | Olivem 1000 | Cetearyl Olivate (and) Sorbitan | 4.00 |
| | | Olivate | |
| | Kaffee Butter | Coffea arabica, Hydrogenated | 4.50 |
| | | Vegetable Oil | |
| | Kakao Butter | Theobroma cacao (Cocoa) | 4.00 |
| | | Seed Butter | |
| | Shea Butter | Butyrospermum parkii | 4.50 |
| | Jojobaöl | Simmondsia chinensis (Jojoba) | 5.00 |
| | | Seed Oil | |
| | Sweet almond Oil | Prunus amygdalus dulcis | 5.00 |
| | | (Sweet Almond) Oil | |
| | DL Tocopherol- | Tocopherol Acetate | 0.50 |
| | acetat | | |
| | Retinol | Retinol Palmitate | 0.50 |
| B | Glycerin | Glycerin | 3.00 |
| | Sisterna L70-C | Aqua, Sucrose Laurate, Alcohol | 3.00 |
| | Euxyl PE 9010 | Phenoxyethanol, Ethylglycerin | 0.80 |
| | Keltrol | Xanthan gum | 0.40 |
| | C*Pharmsorbidex | Sorbitol, Aqua | 1.00 |
| | NC 16205 | | |
| | Vitamin C | Aqua, Ascorbic Acid | 0.30 |
| | Wasser | Aqua | ad 100 |
| C | ALPAFLOR ® | Glycerin, Aqua, Echinacea | 1.00 |
| | Echinacea AO | purpurea (Echinacea) Extract, | |
| | | Potassium Sorbate, Sodium | |
| | | Benzoate | |
| | PEPHA ®- | Aqua, Pullulan, Algae | 4.00 |
| | TIGHT | Extract, Phenoxyethanol, | |
| | | Sodium Benzoate, Potassium | |
| | | Sorbate | |
| | Asiatic Centella | Aqua, Centella asiatica | 0.50 |
| | Extract | | |
| | Aloe vera | Aloe barbadensis Leaf Extract, | |
| | extract | Paraffinum Liquidum | |
| | Composition | Water, Glycerin, Palmitoyl | 3.00 |
| | according | tripeptide-5, Panthenol, | |
| | to table 1 | Marrubium vulgare extract | |
| D | Parfum | Fragrance/Perfum | q.s. |
| | NaOH | Sodium hydroxide | q.s. |

### Example 9

### Silikon-Gel

| Phase | Ingredients | INCI | wt.-% |
|---|---|---|---|
| A | DC 9701 | Dimethicone/Vinyl | 3.00 |
| | | Dimethicone Crosspolymer, | |
| | | Silica | |
| B | Cetiol CC | Dicaprylyl Carbonate | 2.00 |
| | DC 556 Fluid | Phenyl Trimethicone | 2.40 |
| | Lexfeel 7 | Neopentyl Glycol | 3.40 |
| | | Diheptanoate, Isododecane | |
| C | DC 9040 | Cyclopentasiloxane, | Ad 100 |
| | | Dimethicone Crosspolymer | |
| | DC BY 11-030 | Cyclopentasiloxane, | 5.00 |
| | Emulsifier G | PEG/PPG-19/19 Dimethicone | |
| | DC 345 | Cyclopentasiloxane, | 3.00 |
| | | Cyclohexsiloxane | |
| | Euxyl PE 9010 | Phenoxyethanol, Ethylglycerin | 0.80 |
| D | Composition | Water, Glycerin, Palmitoyl | 3.00 |
| | according to | tripeptide-5, Panthenol, | |
| | table 1 | Marrubium vulgare extract | |

### Example 10

### W/Si Emulsion

| Phase | Ingredients | INCI | wt.-% |
|---|---|---|---|
| A | DC 5225C | Cyclopentasiloxane, | 13.60 |
| | | PEG/PPG-18/18 Dimethicone | |
| | | Mineral Oil | 13.60 |
| | DC 345 | Cyclopentasiloxane, | 5.40 |
| | | Cyclohexadecane | |
| B | Water | Aqua | Ad 100 |
| | NaCl | Sodium Chloride | 2.00 |
| | Euxyl PE 9010 | Phenoxyethanol, Ethylglycerin | 0.80 |
| D | Composition | Water, Glycerin, Palmitoyl | 3.00 |
| | according to | tripeptide-5, Panthenol, | |
| | table 1 | Marrubium vulgare extract | |

1. A composition comprising panthenol, an anti-inflammatory plant extract and a collagen synthesis stimulating peptide.

2. The composition according to claim 1, wherein the collagen stimulating peptide is selected from the tripeptide N2-(1-oxohexadecyl)-L-lysyl-L-valyl-L-lysine or a salt thereof and/or the pentapeptide Palmitoyl-Lys-Thr-Thr-Lys-Ser or a salt thereof.

3. The composition according to claim 1 wherein the anti inflammatory plant extract is selected from an extract obtainable from Calendula officinalis, Leontopodium alpinum, Echinacea purpurea, Malva sylvestris, Thymus vulgaris, Peucedanum ostruthium and/or Marrubium vulgare L.

4. The composition according to claim 1, comprising panthenol, a Marrubium vulgare extract and N2-(1-oxohexadecyl)-L-lysyl-L-valyl-L-lysine or a salt thereof.

5. The composition according to claim 4 comprising
(a) at least 10 wt.-% of panthenol
(b) at least 0.005 wt.-% of N2-(1-oxohexadecyl)-L-lysyl-L-valyl-L-lysine or a salt thereof and
(c) at least 0.01 wt.-% of a Marrubium vulgare extract

6. The composition according to claim 4 wherein the N2-(1-oxohexadecyl)-L-lysyl-L-valyl-L-lysine or salt thereof is the bistrifluoroacetate salt of N2-(1-oxohexadecyl)-L-lysyl-L-valyl-L-lysine.

7. The composition according to claim 1 further comprising at least one additional cosmetic additives selected from alcohols and/or water and/or preservatives and whereas the total amount adds up to 100%.

8. A topical preparation comprising an effective amount of a composition according to claim 1 and a cosmetically acceptable carrier.

9. The topical preparation according to claim 8, wherein the effective amount is selected in the range of 1 to 3 wt.-% based on the total weight of the preparation.

10. The topical composition according to claim 8 further comprising at least one further cosmetically active ingredient selected from vitamin E and/or its derivatives, shea butter, algae extract, cocoa butter, aloe extract, elastin and GAG booster, vitamin C (ascorbic acid) and/or its derivatives and/or vitamin A and/or its derivatives.

11. A method of decreasing the actual stretch marks, protecting stretched skin fibers, decreasing the depth of indented surfaces, increasing smooth surfaces, increasing skin thickness and firmness as well as density, increases stimulation of collagen synthesis, moisturizing the skin, reducing inflammation and accelerating skin renewing and wound healing said method comprising the step of applying an effective amount of a topical preparation according to claim 8 to the skin of a subject in need of such a treatment.

12. A method of treatment or co-treatment of stretch marks, said method comprising the step of applying an effective amount of a topical preparation according to claim 8 to the skin of a subject in need of such a treatment.
